(19) United States
(12) Patent Application Publication
(10) Pub. No.: US 2010/0120125 A1
(43) Pub. Date: May 13, 2010

(54) BACTERIAL BIOMINERALIZATION OF CONTAMINANTS

(75) Inventors: PATRICIA A. SOBECKY, Tuscaloosa, AL (US); Robert J. Martinez, Tuscaloosa, AL (US); Martial Taillefert, Atlanta, GA (US); Melanie J. Beazley, Norman, OK (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Georgia Tech Research Corporation, Atlanta, GA (US)

(21) Appl. No.: 12/612,516

(22) Filed: Nov. 4, 2009

Related U.S. Application Data
(60) Provisional application No. 61/111,010, filed on Nov. 4, 2008.

(51) Int. Cl. C12N 1/21; A62D 3/02; C07H 21/04; C12N 15/63; C12M 1/00

(52) U.S. Cl. 435/252.3; 435/262.5; 536/23.2; 435/320.1; 435/289.1

(57) ABSTRACT

The various embodiments of the present disclosure relate generally to bacterial biomineralization of contaminants and, more particularly, to methods, systems and compositions for the biomineralization of soil and water contaminants, such as metals and radionuclides. In one aspect of the present invention, a method for the in situ biomineralization of a soil contaminant comprises: providing a plurality of bacteria to a soil comprising at least one contaminant, wherein at least one bacterium expresses a phosphatase; providing a phosphate-rich material to the soil; reacting at least one bacterium expressing a phosphatase with the phosphate-rich material in the presence of the contaminant; and immobilizing the contaminant in the soil.

### RELATED APPLICATION

This application claims, under 35 U.S.C. §119(e), the benefit of U.S. Provisional Application Ser. No. 61/111,010, filed 4 Nov. 2008, the entire contents and substance of which are hereby incorporated by reference as if fully set forth below.

### STATEMENT REGARDING FEDERALLY SPONSORED RESEARCH

This invention was made with U.S. Government support under Grant No. DE-FG02-04ER63906 awarded by the Department of Energy. The U.S. Government has certain rights in the invention.

### TECHNICAL FIELD OF THE INVENTION

The various embodiments of the present disclosure relate generally to bacterial biomineralization of contaminants and, more particularly, to methods, systems, and compositions for the biomineralization of soil and water contaminants, such as metals and radionuclides.

### BACKGROUND OF THE INVENTION

The remediation of hazardous waste sites, particularly those containing metals and radionuclides, remains one of the most costly environmental challenges currently faced by the United States and other countries. The cost of remediating heavy metal and radionuclide waste in both soils and groundwater has been estimated to be in excess of $600 billion. The U.S. Department of Energy research facilities alone are responsible for the remediation of 40 million cubic meters of soil and 1.7 trillion gallons of contaminated groundwater. Additionally, the U.S. Environmental Protection Agency (EPA) has instituted strict regulations for concentrated animal feeding operations that are annually projected to curb the environmental release of 56 million pounds of phosphorous and 110 million pounds of nitrogen. Thus, the development of innovative approaches to address metal, radionuclide, and nutrient cleanup issues is of both economic and environmental importance.

Previous work, which focused on subsurface and groundwater complexation of soluble uranium, has demonstrated success using abiotic chemical treatments such as zero-valent iron reactive barriers, low solubility hydroxyapatite reactive barriers, sodium phytate, and sodium tripolyphosphate complexing agents. These approaches require the construction of trenches or wells to deliver the chemical of choice into the contaminated subsurface as well as the continuous monitoring of reactive bathers which become clogged and impede hydraulic conductivity once sufficient amounts of uranium complex.

Approaches involving the reductive precipitation of uranium and select heavy metals have been viewed as a cost effective strategy for subsurface remediation. This approach, however, requires the continuous addition of electron donor (acetate, lactate, ethanol, etc.) deep into the subsurface. Unfortunately, this strategy can only occur in anaerobic environments as any infiltration of oxygen will re-solubilize the reductively precipitated uranium and/or heavy metal.

The fate of uranium in a natural environment is governed be a variety of chemical reactions, including reduction/oxidation, sorption/desorption, precipitation/dissolution, and complexation reactions. The two primary oxidation states of uranium are +4 and +6. U(IV) is stable in anaerobic conditions, mainly in the form of the solid mineral uraninite (UO₂₍ₛ₎), but if exposed to dissolved oxygen, uraninite can readily oxidize to the more mobile U(VI). U(VI) dominates in oxidizing conditions as the highly soluble and stable linear uranyl ion UO₂²⁺. Uranyl mobility in natural systems below circumneutral pH is controlled primarily by adsorption, precipitation, and complexation reactions. Uranyl efficiently adsorbs to mineral surfaces such as iron oxides over a range of pH, precipitates as insoluble phosphate minerals, such as autunite (Xⁿ₃₋ₙ(UO₂)₂₋(PO₄)₂ where X can represent H₃O⁺, NH₄⁺, monovalent and divalent metals), and complexes with organic matter, such as humic and fulvic substances. However, above circumneutral pH, uranyl mobility is controlled by carbonates found in most groundwater systems. Carbonate affects the chemical reactions of uranium through the formation of uranyl carbonate complexes. These strong and highly soluble complexes increase the solubility of uranium by limiting mineral adsorption processes and aiding in the oxidation of the U(IV) minerals and dissolution of U(VI) minerals.

Accordingly, there is a need for systems and methods for the mineralization of contaminants, such as metals and radionuclides that operate independently of the redox potential of the contaminants. It is to the provision of such systems and methods for the biomineralization of contaminants that the various embodiments of the present invention are directed.

### BRIEF SUMMARY OF THE INVENTION

The various embodiments of the present disclosure relate generally to bacterial biomineralization of contaminants and, more particularly, to methods, systems and compositions for the biomineralization of soil and water contaminants, such as metals and radionuclides.

An aspect of the present invention comprises a method for the in situ biomineralization of a soil contaminant comprising: providing a plurality of bacteria to a soil comprising at least one contaminant, wherein at least one bacterium expresses a phosphatase; providing a phosphate-rich material to the soil; reacting at least one bacterium expressing a phosphatase with the phosphate-rich material in the presence of the contaminant; and immobilizing the contaminant in the soil. The phosphatase can be selected from one or more of the group consisting of a non-specific phosphohydrolase and a phytase. The phosphate-rich material can comprise an animal manure, such as poultry manure. In one embodiment of the present invention, the phosphatase can comprises an amino acid sequence of SEQ ID NO 2. The contaminant can be selected from one or more of the group consisting of a metal and a radionuclide, such as uranium.

Another aspect of the present invention comprises a substantially purified nucleic acid sequence encoding an enzyme exhibiting phosphatase activity, wherein the nucleic acid sequence is substantially homologous to SEQ ID NO 1. Further, the enzyme exhibiting phosphatase activity can comprise an amino acid sequence that is substantially homologous to SEQ ID NO 2. Yet another aspect of the present invention comprises an expression vector for the enzyme exhibiting phosphatase activity, which can comprise a nucleic acid sequence that is substantially homologous to SEQ ID NO 1 operatively linked to at least one controlled sequence compatible with a suitable bacterial host cell. This expression vector can be transformed into a bacterial cell so that the bacterial cell can express the enzyme exhibiting phosphatase activity in a detectable quantity.

Another aspect of the present invention comprises a system for the production of reactive phosphate comprising: a bioreactor having a chamber; a plurality of bacteria disposed within the chamber, wherein at least one bacterium expresses a phosphatase; and a phosphate-rich material disposed within the chamber, wherein at least one bacterium expressing a phosphatase enzymatically liberates a reactive phosphate group.

The phosphatase can be selected from one or more of the group consisting of a non-specific phosphohydrolase and a phytase. The phosphate-rich material can comprise an animal manure, such as poultry manure. In one embodiment, the plurality of bacteria can comprise Rahnella sp. Y9602. In another embodiment of the present invention, the phosphatase can comprise an amino acid sequence of SEQ ID NO 2. The contaminant can be selected from one or more of the group consisting of a metal and a radionuclide, such as uranium.

A system for the production of reactive phosphate can further comprise a contaminant treatment chamber comprising a contaminant, wherein the system for the production of reactive phosphate is in fluid communication with the contaminant treatment chamber, and wherein the reactive phosphate group liberated by the enzymatic activity of at least one bacterium expressing a phosphatase reacts with the contaminant to precipitate the contaminant. The contaminant is selected from one or more of the group consisting of a metal and a radionuclide, such as uranium.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1 graphically depicts inorganic phosphate production resulting from a 26 day soil slurry incubation of Oak Ridge Field Research Center (ORFRC) contaminated soils supplemented with chicken manure.

FIG. 2 graphically depicts a time course of inorganic phosphate production during a 26 day soil slurry incubation of ORFRC contaminated soils supplemented with chicken manure.

FIG. 3 illustrates a phylogenetic tree derived from a 16S oligonucleotide array, which identifies 1084 unique bacterial strains present in contaminated soils that were supplemented with glycerol-3-phosphate.

FIG. 4 graphically depicts a time course of inorganic phosphate production during a 26 day soil slurry incubation of Rahnella sp. Y9602 supplemented with phytate.

### DETAILED DESCRIPTION

The treatment of hazardous waste sites, particularly those containing metals and radionuclides, remains one of the most costly environmental challenges currently faced by the U.S. and other countries. A potential approach to the immobilization and/or sequestration of these hazardous wastes is the combined use of nonspecific phosphophydrolases (NSAPs) and phytases in naturally occurring subsurface microbial populations to promote the immobilization of divalent metals and radionuclides through the production of insoluble metal phosphate precipitates. Bacterial NSAPs and phytases are microbial phosphatases that demonstrate functionality in acidic pH ranges which are commonly encountered in metal and radionuclide contaminated soils.

Throughout this description, various components can be identified as having specific values or parameters, however, these items are provided as exemplary embodiments. Indeed, the exemplary embodiments do not limit the various aspects and concepts of the present invention as many comparable parameters, sizes, ranges, and/or values can be implemented. The terms “first,” “second,” and the like, “primary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item.

As used herein, the term “biomineralization” refers to the bacterial enzymatic process of liberating a reactive phosphate group from a phosphate source (i.e., an inorganic phosphate molecule and/or an organophosphate molecule) in the presence of a contaminant (e.g., a metal or a radionuclide) to precipitate a water-insoluble contaminant-phosphate complex. Thus, the terms, biomineralization (and mineralization), bioprecipitation (and bioprecipitation), complexation, immobilization, and the like, may be used interchangeably throughout this application.

In one embodiment of the present invention, a method for the in situ biomineralization of a soil contaminant comprises: providing a plurality of bacteria to a soil comprising at least one contaminant, wherein at least one bacterium expresses a phosphatase; providing a phosphate-rich material to the soil; reacting at least one bacterium expressing a phosphatase with the phosphate-rich material in the presence of the contaminant; and immobilizing the contaminant in the soil. A plurality of bacterial and the phosphate-rich material may be provided in situ to the soil by many methods known in the art, including, but not limited to, irrigation of the soil or injection into the soil. In an ex situ method, a contaminant (e.g., industrial effluent, surface or groundwater) can be pumped to a location to be mixed with phosphate-rich medium (e.g., phosphate-rich water), which is generated by a microbial culture that is capable of hydrolyzing an organophosphate source to ultimately precipitate metals and/or radionuclides.

The systems, methods, and compositions of the present invention can comprise a plurality of bacteria. As used herein, the term “plurality” refers to more than one bacterium, and can include one or more types of bacteria. The plurality of bacteria can comprise many soil bacteria, including but not limited to, Proteobacteria, Firmicutes, and Actinobacteria, among others. In an exemplary embodiment of the present invention, the plurality of bacteria can comprise one or more of Rahnella species, Bacillus species, Actinobacter species, Pseudomonas species, Staphylococcus species, Clostria species, Citrobacter species, Xanthomonas species, Klebsiella species, and combinations thereof.

According to the various embodiments of the present invention, the plurality of bacteria includes at least one bacterium expresses a phosphatase. A phosphatase is an enzyme that removes a phosphate group from a substrate by hydrolyzing phosphoric acid monoesters into a phosphate ion and a molecule with a free hydroxyl group. For example, the phosphatases of the present invention can comprise a non-specific phosphophydrolase, a phytase, or a combination thereof.

Such phosphatases can have a variety of phosphate-rich substrates such as various organophosphates, which include but are not limited to, sugar phosphates, phospholipids, alcohols, pesticides, insecticides, and phytate. In an exemplary embodiment of the present invention, the phosphate-rich substrate is an animal manure. The farming industry commonly feed poultry and swine grains and other plant material rich in phytate. Considering that the phytate from these grains is unavailable for bio-absorption, these animals generate manure that is rich in phytate, which has been implicated in eutrophication. Thus, the systems, methods, and compositions of the present invention contemplate the remediation of phytate for the biomineralization of contaminants.

The benefits of utilizing poultry manure, particularly from sources that follow organic farming practices (i.e., feed free of arsenic-containing additives such as roxarsone (Cortinas et al., 2006; Garbarino et al., 2003)), include: (1) the continuous release of phosphate into the soil pore space as a result of stimulation of extant soil and manure microbes capable of hydrolysis of organophosphate-rich manure; and (2) the provision an innovative method for the management of agricultural non-point sources of phosphorus, in order to maintain a clean and safe drinking water quality (U.S. EPA, 1996).

Although not wishing to be bound by any particular theory, it is believed that a non-specific phosphophydrolase and/or a phytase, can hydrolyze an organophosphate (e.g., phytate) to liberate reactive phosphate group. This enzymatically liberated phosphate group can then chemically react with a contaminant to yield and insoluble contaminant-phosphate complex. Suitable contaminants for the systems and methods of the present invention can comprise metals and radionuclides, which can include, but are not limited to, americium, cadmium, cesium, cobalt, copper, iron, lead, mercury, nickel, plutonium, strontium, thorium, uranium, vanadium, and zinc. In an exemplary embodiment of the present invention, the contaminant is uranium. In such embodiments, uranium precipitates with phosphate to form minerals closely related to autunite and meta-autunite groups, which include, but are not limited to, meta-ankoleite (K₂(UO₂)₂₋(PO₄)₂), autunite (Ca(UO₂)₂₋(PO₄)₂), chernikovite (H₂(UO₂)₂₋(PO₄)₂), and saléeite (Mg(UO₂)₂₋(PO₄)₂).

The methods of the present invention may be performed under a variety of conditions. For example, the biomineralization of a contaminant can be performed in both aerobic and anaerobic conditions. Further, the biomineralization of a contaminant can be performed in a variety of pH conditions, ranging from about 3.5 to about 7.5. The methods of the present invention can permit the immobilization of at least about 75%, or about 85%, or preferably at least about 90% or about 95% of a soluble contaminant.

The methods of for the in situ biomineralization of a soil contaminant can be adapted to be performed ex situ. In addition, methods for the biomineralization of a soil contaminant can be used to for the removal of phosphorus from organophosphate waste material.

In aspect of the present invention, a composition comprises a substantially purified nucleic acid sequence encoding an enzyme exhibiting phosphatase activity, wherein the nucleic acid sequence encodes an acid phosphatase or a phytase from a soil bacterium. In an exemplary embodiment, the substantially purified nucleic acid sequence encoding an enzyme exhibiting phosphatase activity is purified from Rahnella sp. Y9602, such as that described in SEQ ID NO 1. The purified nucleic acid sequence encoding an enzyme exhibiting phosphatase activity has substantial homology to SEQ ID NO 1. As used herein, the term “substantial homology” of nucleic acid sequence means that a nucleic acid sequence includes a sequence that has at least 51%, 52%, 53%, 54%, 55%, 56%, 57%, 58%, 59%, 60%, 61%, 62%, 63%, 64%, 65%, 66%, 67%, 68%, or 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, as compared to a reference sequence (e.g., SEQ ID NO 1).

In another aspect of the present invention, a composition comprises an enzyme exhibiting phosphatase activity, wherein the amino acid sequence encodes an acid phosphatase or a phytase from a soil bacterium. In an exemplary embodiment, the amino acid sequence encodes an enzyme exhibiting phosphatase activity, such as the acid phosphatase of Rahnella sp. Y9602, as described in SEQ ID NO 2. The amino acid sequence encodes an enzyme exhibiting phosphatase activity that has substantial homology to SEQ ID NO 2. As used herein, the term “substantial homology” of an amino acid sequence means that an amino acid sequence includes a sequence that has at least 51%, 52%, 53%, 54%, 55%, 56%, 57%, 58%, 59%, 60%, 61%, 62%, 63%, 64%, 65%, 66%, 67%, 68%, or 69%, 70%, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, as compared to a reference sequence (e.g., SEQ ID NO 2).

Another aspect of the present invention comprises a vector for the enzyme exhibiting phosphatase activity comprising the nucleic acid sequence of SEQ ID NO 1. The term “vector” as used herein can refer to a cloning vector or an expression vector.

A cloning vector refers to a plasmid, phage DNA, a cosmid, or other DNA molecule that is able to replicate autonomously in a host cell. A cloning vector is characterized by one or a number of restriction endonuclease recognition sites at which such DNA sequences may be cut in a determinable fashion without loss of an essential biological function of the vector, and into which a DNA fragment (e.g., SEQ ID NO 1) may be spliced in order to bring about its replication and cloning. The cloning vector may further contain a marker suitable for use in the identification of cells transformed with the cloning vector (e.g., an antibiotic resistance marker).

An expression vector is similar to a cloning vector but is capable of expressing a gene which has been cloned into it, after transformation into a host. The cloned gene is usually placed under the control of (i.e., operably linked to) a variety of elements for controlling expression of the gene, including promoter sequences, transcription initiation sequences, enhancer sequences, selectable elements, and reporter genes. Examples of suitable expression vectors include, but are not limited to, plasmids, phagemids, cosmids, artificial chromosomes, such as a yeast artificial chromosome (YAC), a bacterial artificial chromosome (BAC), or a P1-derived artificial chromosome (PAC), and bacteriophages, such as lambda phage or M13 phage.

Another aspect of the present invention comprises a bacterial cell transformed with the expression vector encoding SEQ ID NO 1, wherein the bacterial host cell can express the enzyme exhibiting phosphatase activity in a detectable quantity. The bacterial cell can comprise many soil bacteria, including but not limited to, Proteobacteria, Firmicutes, and Actinobacteria, among others. In an exemplary embodiment of the present invention, the bacterial can comprise one or more of Rahnella species, Bacillus species, Actinobacter species, Pseudomonas species, Staphylococcus species, Clostria species, Citrobacter species, Xanthomonas species, Klebsiella species, and combinations thereof.

A number of procedures exist for the introduction of DNA into those bacteria. A person of ordinary skill in the art could readily ascertain the suitable transformation method for the appropriate host bacterium. For example, a very simple, moderately efficient transformation procedure involves re-suspending log-phase bacteria in ice-cold 50 mM calcium chloride at about 10¹⁰ bacteria/ml and keeping them ice-cold for about 30 min. Plasmid DNA (0.1 mg) is then added to a small aliquot (0.2 ml) of these now competent bacteria, and the incubation on ice continued for a further 30 minutes, followed by a heat shock of 2 minutes at 42° C. The bacteria are then usually transferred to nutrient medium and incubated for some time (30 minutes to 1 hour) to allow phenotypic properties conferred by the plasmid to be expressed, (e.g., antibiotic resistance commonly used as a selectable marker for plasmid-containing cells). Protocols for the production of competent bacteria have been described (Hanahan (J. Mol. Biol. 166: 557-580 (1983); Liu et al., Bio Techniques 8:21-25 (1990); Kushner, In: Genetic Engineering: Proceedings of the International Symposium on Genetic Engineering, Elsevier, Amsterdam, pp. 17-23 (1978); Norgard et al., Gene 3:279-292 (1978); Jessee et al., U.S. Pat. No. 4,981,797); Maniatis et al., Molecular Cloning, A Laboratory Manual, Cold Spring Harbor Press, Cold Spring Harbor, N.Y. (1982).

Another rapid and simple method for introducing genetic material into bacteria is through electroporation (Potter, Anal. Biochem. 174: 361-73 (1988)). This technique is based upon the original observation by Zimmerman et al. (J. Membr. Biol. 67: 165-82 (1983)) that high-voltage electrical pulses can induce cell plasma membranes to fuse. Subsequently, it was found that when subjected to electric shock (typically a brief exposure to a voltage gradient of 4000-16000 V/cm), the bacteria can take up exogenous DNA from the suspending solution, apparently through holes momentarily created in the plasma membrane. A proportion of these bacteria become stably transformed and can be selected if a suitable marker gene is carried on the transforming DNA (Newman et al., Mol. Gen. Genetics 197: 195-204 (1982)).

Bacterial cells are also susceptible to transformation by liposomes (Old and Primrose, In: Principles of Gene Manipulation: An Introduction to Gene Manipulation, Blackwell Science (1995)). A simple transformation system has been developed which makes use of liposomes prepared from cationic lipids. Small unilamellar (single bilayer) vesicles are produced and DNA in solution spontaneously and efficiently complexes with these liposomes. The positively-charged liposomes not only complex with DNA, but also bind to bacteria and are efficient in transforming them, probably by fusion with the cells.

Another aspect of the present invention comprises a system for the production of reactive phosphate comprising: a bioreactor having a chamber; a plurality of bacteria disposed within the chamber, wherein at least one bacterium expresses a phosphatase; and a phosphate-rich material disposed within the chamber, wherein at least one bacterium expressing a phosphatase enzymatically liberates a reactive phosphate group. In an exemplary embodiment, a bacterium of the system comprises Rahnella sp. Y9602.

This system is designed to enzymatically remediate phosphate-rich materials (e.g., poultry manure, swine manure, or combinations thereof) into reactive phosphate groups. The resulting reactive phosphate groups can be used to immobilize contaminants in a soil or contaminants in water. For example, the system can further comprise a contaminant treatment chamber in fluid communication with the system for the production of reactive phosphate. The contaminant treatment chamber can contain a variety of media. As used herein, the term “medium” can comprise many media, including but not limited to, a fluid, liquid, solid, solution, suspension, emulsion, gas, vapor, gel, dispersion, a flowable material, a multiphase material, or combination thereof. In an exemplary embodiment of the present invention, the medium can be soil, rock, industrial waste, groundwater, or a combination comprising at least two of the foregoing, among others.

Thus, according to the various embodiments of the present invention, the system for the production of reactive phosphate is in fluid communication with the contaminant treatment chamber, wherein the reactive phosphate group liberated by the enzymatic activity of at least one bacterium expressing a phosphatase reacts with the contaminant in the contaminant treatment chamber to precipitate the contaminant. Of course, the contaminant treatment chamber and the bioreactor chamber can be the same chambers or they can be different chambers.

All patents, patent applications, and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure. Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

### EXAMPLES

### Example 1

### Bacterial Enzymatic Hydrolysis of Poultry Manure

Subsurface strains and growth conditions. Metal-resistant subsurface strains Arthrobacter spp. (X34, V45, AA20), Bacillus spp. (Y7, X18, Y9-2) and Rahnella spp. (Y9602, Y4, Y29) were previously isolated from radionuclide- and metal-contaminated subsurface soils collected from the Oak Ridge Field Research Center (ORFRC) as described in Martinez and colleagues (2006). Detailed geology, chemistry and site descriptions are available on the DOE Environmental Remediation Sciences Program website (http://www.esd.ornl.gov/nabirfrc/). Strains were isolated from soil core samples as described in Martinez and colleagues (2006) from sites where the saturated zones contained elevated uranium, other radionuclides and heavy metals (Brooks, 2001). Strain identification was previously confirmed by 16S rDNA phylogeny (Martinez et al., 2006). Media used to identify strains with constitutive phosphatase activity were TPMG (Riccio et al., 1997) and TP-MUP modified from Adcock and Saint (2001). The modified TP-MUP consisted of 20 g tryptose, 5 g sodium chloride, 2.5 g disodium phosphate, 2 g dextrose, 85 mg 4-methylumbelliferyl phosphate, and 15 g agar per liter. Plates were incubated at 30° C. for 24 h with the exception of TPMG (36 h). Cells were grown in pH-buffered SGW consisting of 50 mM MES (pH 5.5), 2 μM FeSO₄, 5 μM MnCl₂, 8 μM Na₂MoO₄, 0.8 mM MgSO₄, 7.5 mM NaNO₃, 0.4 mM KCl, 7.5 mM KNO₃, 0.2 mM Ca(NO3)₂, and 10 mM glycerol-3-phosphate (Sigma) as the sole carbon and phosphorus source. Nutrient broth (NB) agar (3 g beef extract, 5 g peptone, 15 g agar per liter) was used for the maintenance of the strains. All strains were incubated at 30° C., and liquid cultures were shaken at 200 r.p.m.

Phosphatase Activity Assay. Soil slurry incubations were adapted from recently published work (Martinez et al., 2007; Beazley et al., 2007). Briefly, all incubations were conducted in sterilized acid washed 500 ml glass Erlenmeyer flasks with slurry volume adjusted to 125 ml with synthetic groundwater (SGW), pH 5.5. SGW was composed of: 50 mM MES (pH 5.5), 2 μM FeSO₄, 5 μM MnCl₂, 8 μM Na₂MoO₄, 0.8 mM MgSO₄, 7.5 mM NaNO₃, 0.4 mM KCl, 7.5 mM KNO₃, 0.2 mM Ca(NO₃)₂, and 10 mM glycerol (EMD Chemicals). The following components were added to each flask: contaminated soils (2 g), poultry manure including litter pine shavings (2.5 g) and pH buffered SGW to yield a final volume of 125 ml. All incubations were maintained at 30° C. and shaken at 200 rpm for 26 days using a Lab-Line Incubator-Shaker Model 3525. Phosphate concentrations were determined by spectrophotometry (Murphy and Riley, 1962).

Table 1 shows the inorganic phosphate accumulation of ORFRC contaminated soil slurries supplemented with 2.5 g chicken manure.

TABLE 1

| | |
|---|---|
| Time | [PO₄³⁻] - μM |

| (day) | FW 120-06-00 | FW 120-08-00 |
|---|---|---|
| 0 | 182 | 154 |
| 2 | 223 | 99 |
| 4 | 300 | 145 |
| 6 | 310 | 175 |
| 8 | 343 | 237 |
| 10 | 205 | 277 |
| 12 | 365 | 271 |
| 14* | 427 | 274 |
| 16 | 182 | 79 |
| 18 | 310 | 188 |
| 20 | 346 | 235 |
| 22 | 368 | 246 |
| 24 | 359 | 258 |
| 26 | 359 | 297 |

*10 mM glycerol supplementation of soil slurries

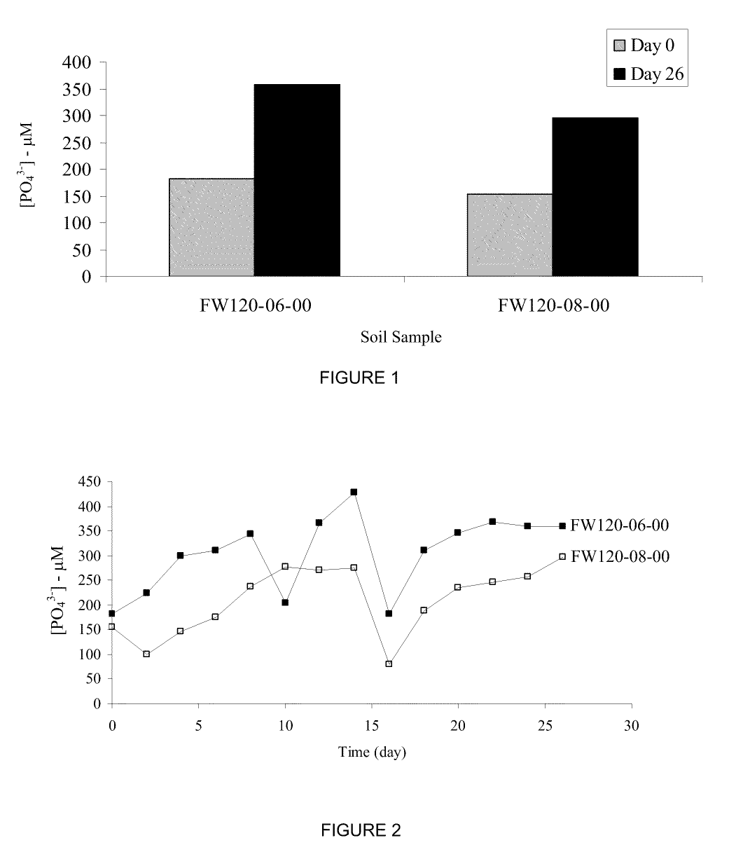

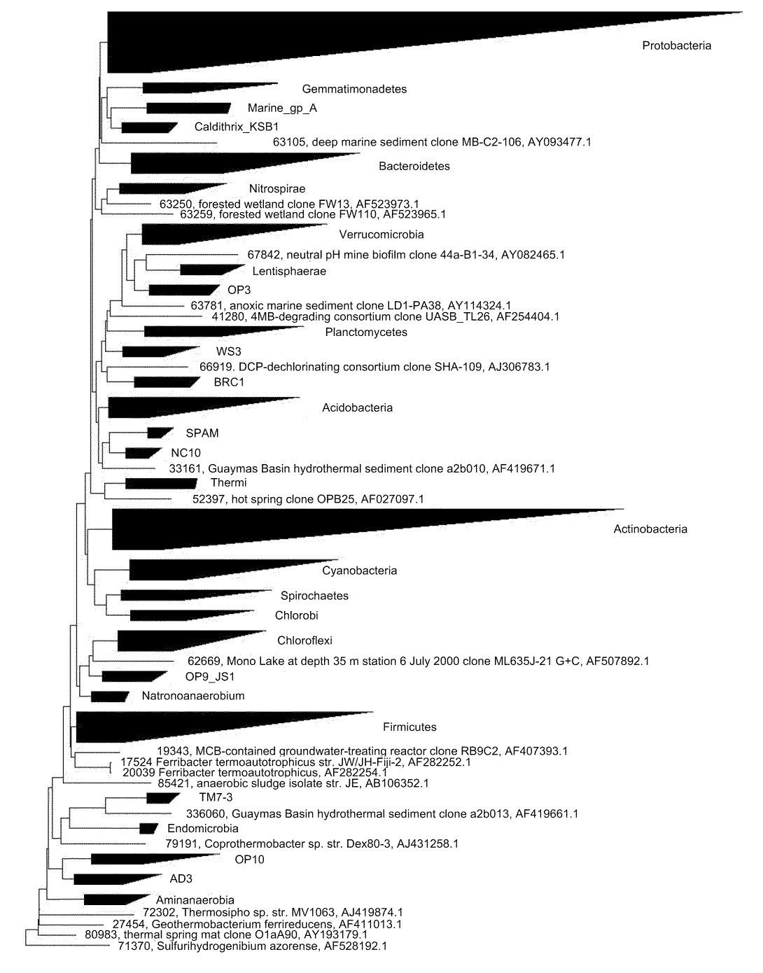

FIG. 2 displays a time course of inorganic phosphate production during a 26 day soil slurry incubation of ORFRC contaminated soils supplemented with 2.5 grams of chicken manure. These data demonstrate that chicken manure supplemented to metal and radionuclide contaminated soils provide sufficient concentrations of inorganic reactive phosphate as well as organophosphates which can be hydrolyzed over the course of a 26 day experiment (Table 1 and FIGS. 1-2). Utilizing this inorganic reactive phosphate, aerobic uranium precipitation can be promoted by the activity of microbial non-specific acid phosphatase enzymes in contaminated subsurface soils. Further, these data indicate that poultry manure can be used as an organophosphorus source that is suitable for promoting the aerobic precipitation of heavy metals and radionuclides in contaminated soils.

### Example 2

### Identification of Bacterial Population of Contaminated Soils.

The total diversity of organisms present in contaminated soils that were supplemented with glycerol-3-phosphate was assessed utilizing 16S rDNA analysis. FIG. 3 illustrates a phylogenetic tree representing 1084 unique bacterial strains. The size of the triangles correlate to the number of organisms detected within a given phylum or newly established grouping

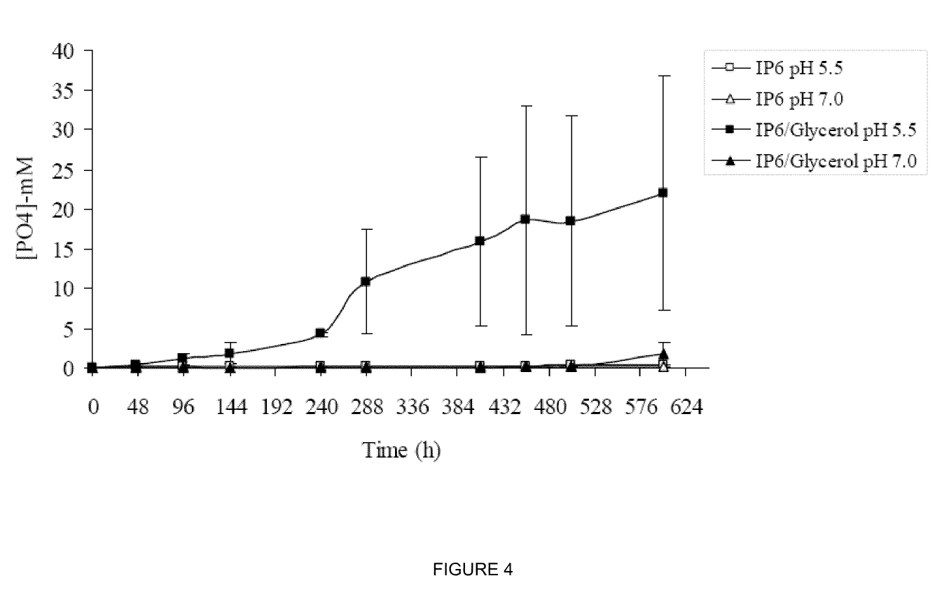

This is the approach allows for the rapid identification of bacterial strains that have the ability to hydrolyze organophosphates in a given environment. One such example is Rahnella sp. Y9602. This Rahnella strain was incubated in minimal media at pH 5.5. or pH 7.0 supplemented with phytate (also referred to as inositol hexakisphosphate (IP6)) for 26 days in the presence and absence of the carbon source, glycerol (FIG. 4). The data indicates that the Rahnella strain supplemented with glycerol has an enzymatic optima at pH 5.5 for phytate when compared the pH 7.0 incubation. The total amount of phosphate that can be hydrolyzed from the phytate in these incubations ranges from about 20 mM to about 100 mM.

Given the ability of the Rahnella strain to hydrolyze organophosphates, the non-specific acid phosphatase gene of is Rahnella sp. Y9602 was identified via PCR amplification of an excised portion of the genome using an inverse PCR technique. The primers used for inverse PCR are: Y9602A Forward 5′-GGTAATTTGCGGCTACCA-3′ (SEQ ID NO 3) and Y9602A Reverse 5′-CTGTTCATACATGGCCTGAT-3′ (SEQ ID NO 4). This provided the DNA flanking the full length gene, and an open reading frame (ORF) prediction program was used to identify the putative start and stop codons. The ORF for the non-specific acid phosphatase gene of is Rahnella sp. Y9602 is provided as SEQ ID NO 1, and the protein that can be translated from this sequence is provided as SEQ ID NO 2.

SEQUENCES
SEQ ID NO. 1-Rahnella sp. Y9602 acid phosphatase
nucleic acid sequence
ATGAAAATTATTACTACTTTTTGCCTCGCCAGTCTTTTCACTGTCAATGC
GTTTGCCCTGACCGGCAACGATGCGACCACCAAGCCGGATCTCTACTACT
TAAAAAACGATCAGGCGATTAACAGTCTGGCGCTGCTTCCGCCACCACCC
GCAGTGGGCAGTATTGCTTTTTTGAACGATCAGGCCATGTATGAACAGGG
TCGTCTGCTGCGCTCAACTGAACGTGGCAAACTGGCGGCAGAAGATGCCA
ACCTGAGTGCGGGTGGTGTCGCGAACGCCTTCTCCGGTGCCTTTGGTTCG
CCAATCACCGCCAAAGACACCCCGGAACTGCACAAACTGCTGACCAATAT
GATTGAAGATGCAGGTGATCTGGCGACGCGTTCCGCCAAAGAAAAGTACA
TGCGCATTCGTCCGTTTGCCTTCTACGGCGTGCCGACCTGTAACACCACC
GAGCAGGATAAACTGTCGAAAAACGGTTCGTATCCTTCCGGCCACACCTC
AATTGGCTGGGCCACCGCGCTGGTGCTGACCGAAATTAACCCGCAGCGCC
AGGACCAAATCCTGCAACGCGGTTTCGATTTAGGTCAGAGCCGGGTAATT
TGCGGCTACCACTGGCAAAGTGATGTCGATGCCGCGCGCATCGTCGGTTC
CGCAGTAGTGGCAACCCTGCATACCAACTCTGCTTTCCAGCAGCAGTTGC
AGAAAGCCAAAGAAGAGTTTGCGAAGCAGCATCCGTAA
SEQ ID NO 2-Rahnella sp. Y9602 acid phosphatase
amino acid sequence
MKIITTFCLASLFTVNAFALTGNDATTKPDLYYLKNDQAINSLALLPPPP
AVGSIAFLNDQAMYEQGRLLRSTERGKLAAEDANLSAGGVANAFSGAFGS
PITAKDTPELHKLLTNMIEDAGDLATRSAKEKYMRIRPFAFYGVPTCNTT
EQDKLSKNGSYPSGHTSIGWATALVLTEINPQRQDQILQRGFDLGQSRVI
CGYHWQSDVDAARIVGSAVVATLHTNSAFQQQLQKAKEEFAKQHP
SEQ ID NO 3-Rahnella sp. Y9602 Forward Primer
GGTAATTTGCGGCTACCA
SEQ ID NO 4-Rahnella sp. Y9602 Reverse Primer
CTGTTCATACATGGCCTGAT

### REFERENCES

Arey, J. S., Seaman, J. C. and Bertsch, P. M. 1999 Immobilization of uranium in contaminated sediments by hydroxyapatite addition. Environmental Science & Technology 33: 337-342.

Beazley, M. J., Martinez, R. J., Webb, S. M., Sobecky, P. A. and Taillefert, M. 2007. The effect of enzymatic activity, pH, and metal-to-ligand ratio on the biomineralization of U(VI) in aerobic conditions. Environmental Science and Technology 41: 5701-5707.

Cortinas, I., Field, J. A., Kopplin, M., Garbarino, J. R., Gandolfi, A. J. and Sierra-Alvarez, R. 2006. Anaerobic biotransformation of roxarsone and related n-substituted phenylarsonic acids. Environmental Science & Technology 40: 2951-2957.

Garbarino, J. R., Bednar, A. J., Rutherford, D. W., Beyer, R. S. and Wershaw, R. L. 2003. Environmental fate of roxarsone in poultry litter. I. Degradation of roxarsone during composting. Environmental Science & Technology 37: 1509-1514.

Martinez, R. J., Beazley, M. J., Taillefert, M., Arakaki A. K., Skolnick, J. and Sobecky, P. A. 2007. Effect of uranium (VI) on lead resistant bacteria isolated from radionuclide- and metal-contaminated subsurface soils. Environmental Microbiology (accepted).

Martinez, R. J., Wang, Y. L., Raimondo, M. A., Coombs, J. M., Barkay, T., and Sobecky, P. A. (2006) Horizontal gene transfer of PIB-type ATPases among bacteria isolated from radionuclide- and metal-contaminated subsurface soils. Appl Environ Microbiol 72: 3111-3118.

Murphy, J., and Riley, J. P. 1962. A modified single solution method for determination of phosphate in natural waters. Analytica Chimica Acta 26: 31-36.

Phillips, D. H., Gu, B., Watson, D. B., Roh, Y., Liang, L and Lee, S. Y. 2000. Performance evaluation of a zerovalent iron reactive barrier: Mineralogical characteristics. Environmental Science & Technology 34: 4169-4176.

U.S. Environmental Protection Agency. 1996. Environmental indicators of water quality in the United States. EPA 841-R-96-002. U.S. EPA, Office of Water (4503F), U.S. Govt. Printing Office, Washington, D.C.

Wellman, D. M., Icenhower, J. P. and Owen, A. T. 2006. Comparative analysis of soluble phosphate amendments for the remediation of heavy metal contaminants: Effect on sediment hydraulic conductivity. Environmental Chemistry 3: 219-224.

What is claimed is:

1. A method for the in situ biomineralization of a soil contaminant comprising:
providing a plurality of bacteria to a soil comprising at least one contaminant, wherein at least one bacterium expresses a phosphatase;
providing a phosphate-rich material to the soil;
reacting at least one bacterium expressing a phosphatase with the phosphate-rich material in the presence of the contaminant; and
immobilizing the contaminant in the soil.

2. The method for the in situ biomineralization of a soil contaminant of claim 1, wherein the phosphatase is selected from one or more of the group consisting of a non-specific phosphohydrolase and a phytase.

3. The method for the in situ biomineralization of a soil contaminant of claim 1, wherein the phosphate-rich material comprises an animal manure.

4. The method for the in situ biomineralization of a soil contaminant of claim 1, wherein the animal manure is poultry manure.

5. The method for the in situ biomineralization of a soil contaminant of claim 4, wherein the phosphatase comprises an amino acid sequence of SEQ ID NO 2.

6. The method for the in situ biomineralization of a soil contaminant of claim 1, wherein the contaminant is selected from one or more of the group consisting of a metal and a radionuclide.

7. The method for the in situ biomineralization of a soil contaminant of claim 1, wherein the contaminant is uranium.

8. A substantially purified nucleic acid sequence encoding an enzyme exhibiting phosphatase activity, wherein the nucleic acid sequence is substantially homologous to SEQ ID NO 1.

9. The substantially purified nucleic acid molecule of claim 8, wherein the enzyme exhibiting phosphatase activity comprises an amino acid sequence that is substantially homologous to SEQ ID NO 2.

10. An expression vector for the enzyme exhibiting phosphatase activity comprising the nucleic acid sequence of claim 8, operatively linked to at least one controlled sequence compatible with a suitable bacterial host cell.

11. A bacterial cell transformed with the expression vector of claim 10, wherein the bacterial cell can express the enzyme exhibiting phosphatase activity in a detectable quantity.

12. A system for the production of reactive phosphate comprising:
a bioreactor having a chamber;
a plurality of bacteria disposed within the chamber, wherein at least one bacterium expresses a phosphatase; and
a phosphate-rich material disposed within the chamber,
wherein at least one bacterium expressing a phosphatase enzymatically liberates a reactive phosphate group.

13. The system for the production of reactive phosphate of claim 12, wherein the phosphatase is selected from one or more of the group consisting of a non-specific phosphohydrolase and a phytase.

14. The system for the production of reactive phosphate of claim 12, wherein the phosphate-rich material comprises an animal manure.

15. The system for the production of reactive phosphate of claim 12, wherein the animal manure is poultry manure.

16. The system for the production of reactive phosphate of claim 12, wherein the plurality of bacteria comprises Rahnella sp. Y9602.

17. The system for the production of reactive phosphate of claim 12, wherein the phosphatase comprises an amino acid sequence having substantial homology to SEQ ID NO 2.

18. The system for the production of reactive phosphate of claim 12, the system further comprising a contaminant treatment chamber comprising a contaminant, wherein the system for the production of reactive phosphate is in fluid communication with the contaminant treatment chamber, and wherein the reactive phosphate group liberated by the enzymatic activity of at least one bacterium expressing a phosphatase reacts with the contaminant to precipitate the contaminant.

19. The system for the production of reactive phosphate of claim 18, wherein the contaminant is selected from one or more of the group consisting of a metal and a radionuclide.


SEQUENCE LISTING (4 sequences)

SEQ ID NO: 1 - DNA, 738 bp, Rahnella sp. Y9602
atgaaaattattactactttttgcctcgccagtcttttcactgtcaatgcgtttgccctgaccggcaacgatgcgaccaccaagccggatctctactacttaaaaaacgatcaggcgattaacagtctggcgctgcttccgccaccacccgcagtgggcagtattgcttttttgaacgatcaggccatgtatgaacagggtcgtctgctgcgctcaactgaacgtggcaaactggcggcagaagatgccaacctgagtgcgggtggtgtcgcgaacgccttctccggtgcctttggttcgccaatcaccgccaaagacaccccggaactgcacaaactgctgaccaatatgattgaagatgcaggtgatctggcgacgcgttccgccaaagaaaagtacatgcgcattcgtccgtttgccttctacggcgtgccgacctgtaacaccaccgagcaggataaactgtcgaaaaacggttcgtatccttccggccacacctcaattggctgggccaccgcgctggtgctgaccgaaattaacccgcagcgccaggaccaaatcctgcaacgcggtttcgatttaggtcagagccgggtaatttgcggctaccactggcaaagtgatgtcgatgccgcgcgcatcgtcggttccgcagtagtggcaaccctgcataccaactctgctttccagcagcagttgcagaaagccaaagaagagtttgcgaagcagcatccgtaa

SEQ ID NO: 2 - protein, 245 aa, Rahnella sp. Y9602
MKIITTFCLASLFTVNAFALTGNDATTKPDLYYLKNDQAINSLALLPPPPAVGSIAFLNDQAMYEQGRLLRSTERGKLAAEDANLSAGGVANAFSGAFGSPITAKDTPELHKLLTNMIEDAGDLATRSAKEKYMRIRPFAFYGVPTCNTTEQDKLSKNGSYPSGHTSIGWATALVLTEINPQRQDQILQRGFDLGQSRVICGYHWQSDVDAARIVGSAVVATLHTNSAFQQQLQKAKEEFAKQHP

SEQ ID NO: 3 - DNA, 18 bp, Rahnella sp. Y9602
ggtaatttgcggctacca

SEQ ID NO: 4 - DNA, 20 bp, Rahnella sp. Y9602
ctgttcatacatggcctgat